(19) United States
(12) Patent Application Publication
(10) Pub. No.: US 2008/0249894 A1
(43) Pub. Date: Oct. 9, 2008

(54) Product samples, methods for producing product samples and methods for distribution of product samples

(76) Inventor: Barry Madel, Hudson, OH (US)

Correspondence Address: EDWIN OLDHAM, 400 Park Shore Drive #200, Naples, FL, 34103, US

(21) Appl. No.: 11/982,721

(22) Filed: Nov. 2, 2007

Related U.S. Application Data
(60) Provisional application No. 60/856,249, filed on Nov. 2, 2006.

(51) Int. Cl. G06Q 30/00; G01N 33/00

(52) U.S. Cl. 705/27; 73/866

(57) ABSTRACT

The invention is directed to a device and methods for assisting in the selection of finishing materials or products such as countertop materials, flooring materials or products or a wide variety of other finish materials or products that are not conducive to forming product samples having desired characteristics. The devices may include a base substrate having an image of a finish material or product provided thereon, and a layer of material which provides a predetermined visual appearance shift in the image associated with the base substrate to make a laminate. The top surface of the laminate may be provided with a surface texture to form a product sample which closely simulates the color, pattern, texture and/or gloss, reflectance or light absorption associated with an actual predetermined finish material or product. The invention also relates to a method of manufacturing a product sample comprising the steps of providing a base substrate and forming an image on the substrate relating to a predetermined color and design of a finishing material or product. A layer of material is applied to the substrate to cause a predetermined visual appearance shift in the image formed on the base substrate to form a laminate. The top surface of the laminate is provided with predetermined surface texturing to provide a sample which closely simulates the color, pattern and texture of a predetermined finishing material or product. The present invention also relates to offering product samples via the Internet or other distribution channels not presently used to provide the user with desired product samples. The invention provides significant advantages in the selection of finishing materials for the consumer, or may be provided to architects and designers in various forms to facilitate use.

### RELATED APPLICATIONS

This relates to and claims priority to U.S. Provisional Patent Application Ser. No. 60/856,249 filed Nov. 2, 2006, which is hereby incorporated by reference herein.

### BACKGROUND

The invention relates to products and methods for manufacturing product samples, and methods for distribution of product samples. The product samples may be made to emulate products or materials such as various forms of surface coverings, counter top materials, floor coverings, wall coverings, cabinet wood or finishes, ceramic tiles, solid surface materials, door or window treatments, decorating treatments or the like. These may include for example, high pressure laminates for use on countertops, flooring or furniture, solid surface materials used for countertops, floor coverings including wood, tile and carpet, metal laminates, veneers, stone, and many other such products. The appearance of such products is an important consideration, and many such products are designed to emulate natural materials or may be formed of natural materials to provide a desired appearance. These appearances include, for example, ceramic tile, stone, granite, brick, sandstone, cork, wood, and a wide variety of other finishes or appearances. Further, installation may further require in some cases, the addition of textured lines or grout lines, which also add to the overall appearance. Presently, product samples provided of such products are deficient in many respects, being costly, and not adequately providing the user with the ability to envision the product for a particular use or area as would be helpful.

During the process of designing or remodeling a home, business or the like, architects and professional designers, all the way to the average consumer, desire the ability to view and determine the desirability of various product appearances based on colors, textures and other characteristics of the products or materials. This presents a problem for the manufacturer in supplying suitable product samples to assist the consumer in selecting the products for purchase, as many of such products are difficult to display in a suitable manner. Further, there is a very large selection of styles, types, colors and textures of such products and materials. The materials themselves are also generally not conducive to this process. Such materials may be supplied for use in roll form, sheet form, or other forms which make it difficult to display to a potential consumer. Similarly, many such materials are heavy, bulky, or otherwise difficult to handle in their manufactured form, and thereby difficult to handle and display to potential consumers.

To assist in the process of designing or remodeling for example, manufacturers have attempted to provide small product samples that can be viewed by a consumer. Such product samples are formed of the actual materials, and depending on the product or material, is generally a small sized sample, which is either simply separately available to the consumer, or displayed in some cases with multiple samples mounted on a board or the like. For example, high pressure laminate materials used for countertops, such as produced by manufacturers like Formica Corporation or Wilsonart International, are formed as a sheet which is shipped and delivered to an installation site or a fabricator. In such a form, the consumer cannot easily see the material or compare different colors or styles. Therefore such manufacturers have resorted to making small samples of the sheet material, which are distributed to architects and designers or other distribution outlets, and retail outlets for use in comparison of these products. Such product samples are generally small chips of countertop products or materials, which are selectively secured on a key ring or chain, or hung on a rack outfitted with hooks for example. As the samples are the actual products, the cost associated with producing and preparing such samples is significant. Further, although providing some indication of the appearance of the product, such samples may still make it difficult to envision the appearance of the surface on which the products are to be installed. With such materials for example, the manufacturer may provide different finish surface appearances with a similar base product, such as natural, brushed, matte, gloss, sparkle, powdered, etched, quilted, crystal or a wide variety of other finishes that change the appearance of the final product. The small sized samples simply do not allow the consumer to effectively distinguish these distinctions. Similar materials, such as some floor covering materials, wall covering materials, or the like, have similar problems in terms of allowing the consumer the ability to adequately envision the material for use in a larger area. It would be desirable to provide the ability to provide product samples which would allow the consumer to properly envision these different product characteristics more easily.

For other types of products, such as granite, quartz, other stone surfaces, solid surface materials, tile flooring, wood flooring, carpeting or a wide variety of other similar materials, providing product samples again poses significant problems. Such materials are thicker or larger and heavier, and necessitate attempts by manufacturers to again provide small samples, but in a form which can be more easily handled, such as by mounting such small samples on a large board or binding samples together in a bundle. Such displays are difficult to handle by the architects and designers, let alone the actual consumer, and the user again must attempt to envision the product or material over a large area in which they are to be used, but using only a small sample. The difficulty in handling, as well as the significant costs of shipping and maintaining such samples would make an alternative desirable for both the manufacturer and consumer. These types of products also have a three dimensional type of surface appearance due to the use of different materials in a matrix, or the characteristics of the natural product. It would be desirable to provide an simulated product sample that had the appearance of the actual product, or other types of sheet type products having a surface image that provides a three-dimensional type appearance.

In the design process of any residential, business or public facility for example, the architect, designer or consumer would benefit from the ability to properly envision the finish materials used in the construction. It would be desirable to provide the ability for the display of a simulation of the actual materials for enhancing the selection process and avoiding the mistake of choosing a product that doesn't meet the expectations of the consumer. It would also be desirable to provide the ability for the architect, designer or consumer to envision the color scheme, texture and coordination of surface finishes in a room or environment, such as the countertop surfacing, floor surfacing, wall surfacing, cabinetry, even appliances, furniture or other accessories. It would also be desirable to provide a product sample system for such materials that enables the user to cost effectively use larger product samples to evaluate the choices available.

### SUMMARY OF THE DISCLOSURE

The invention overcomes the problems of the prior approaches to handling product samples for many types of finishing products as noted above. The invention provides a product and process of manufacturing a product which is a simulation of the actual finishing products, to assist in the selection of such products and materials, such as countertop surfacing, floor surfacing, wall surfacing, cabinetry, fabrics on furnishings or other surfacing or finishing materials.

The invention also provides product samples simulating the finishing materials and products, that significantly reduce costs of manufacture, shipping and handling of product samples, and allows a manufacturer to further facilitate selection of such products or materials with the use of high quality printed graphics or advertising in association with the product sample.

Other aspects of the present invention relate to providing a display of product samples relating to various colors, designs or textures of products or materials to provide assistance to a consumer in the selection of the type, style and color of the product or material.

These advantages of the invention are provided by a device for assisting in the selection of finishing materials comprising a base substrate having an image of a finishing material provided thereon, and a layer of material which provides a predetermined visual appearance shift in the image of the base substrate to closely simulate the finished product or material appearance. The top surface of the product sample may be provided with a surface texture to closely simulate the perceived color, pattern and texture of a predetermined finishing material or product. In another aspect, the device has a multi-layer type construction to provide a three-dimensional type appearance.

The invention also relates to a method of manufacturing a product sample comprising the steps of providing a base substrate and forming an image on the substrate relating to a predetermined color and design of a finishing material or product. A layer of material is applied to the substrate to cause a predetermined visual appearance shift in the image formed on the base substrate to form a laminate. The top surface of the laminate is provided with predetermined surface texturing to provide a sample which closely simulates the color, pattern and texture of a predetermined finishing material or product. In another aspect, the product sample may have an overlayer of a clear material, with a printed layer having at least one non-opaque area associated therewith. Below the printed layer, the device may include one or more film layers and an opaque backing layer. The arrangement of the one or more film layers below the non-opaque areas in the printed layer provide a three-dimensional type of appearance.

The present invention may be used in retail environments to provide design assistance to consumers during the selection of finishing materials, or may be provided to architects and designers in various forms to facilitate use. Alternatively, due to the nature of the product sample device according to embodiments of the present invention, the product sample devices may be distributed through other channels, such as via an online Internet store, where consumers can order and receive product sample devices through the mail in an easy and convenient manner.

It is to be understood that both the foregoing general description and the following detailed description are exemplary and are intended to provide further explanation of the invention as claimed. The accompanying drawings illustrate and provide a further understanding of the embodiments of the devices or products according to the invention and method of the claimed invention.

### BRIEF DESCRIPTION OF THE DRAWINGS

FIG. 1 is a perspective view of an example of the product sample device according to the invention;

FIG. 2. is an enlarged side view of an example of the product sample device according to the invention;

FIG. 3 is a perspective view of a further example of the product sample device according to the invention;

FIG. 3A. is an enlarged side view of a further example of the product sample device according to the invention;

FIG. 4. is an enlarged side view of a further example of the product sample device according to the invention

FIG. 5 provides a plan view of a further example of a sample display device according to the invention;

FIG. 6 is a plan view of a further example of the product sample device formed in a sheet and being perforated to allow for folding and shipment in a flat position, and to selectively provide smaller portions of the sheet for use;

FIG. 7 shows a schematic illustration of a system for distribution of product sample devices according to an embodiment of the invention; and

FIG. 8 is a flow diagram of a method for manufacture and distribution of product sample devices according to the invention.

### DETAILED DESCRIPTION OF THE DRAWINGS

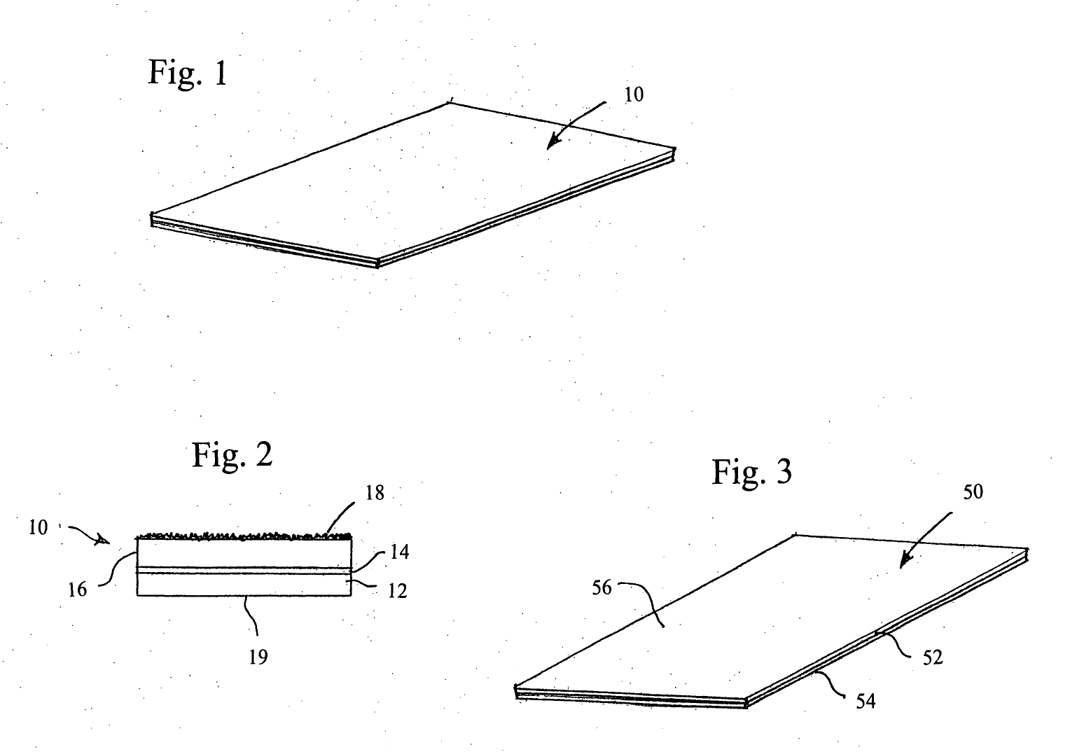

The disclosure is directed to product sample devices and displays, as well as methods of manufacturing such devices, and methods for distribution of such products. In accordance with the invention, the terms “finish products or materials” is intended to include a wide variety of products and materials as mentioned above, including but not limited to countertop materials, flooring materials, wall covering materials, fabrics, furnishing materials, cabinet woods and finishes, stone products, solid surface types of products, flooring tiles, and other similar materials which comprise surface enhancements having attributes of coloring, patterning, surface treatments and the like, and wherein the products or materials are not of a form to allow desired product samples or displays of product samples to be provided for use by a consumer. Turning to FIG. 1, the product sample device 10 according to an embodiment provides a sample sheet that has an appearance which closely simulates the appearance of an actual finishing product or material. The device 10 may be configured to have desired dimensions which can vary greatly to provide flexibility of use. The device is generally formed as a sheet of laminated layers of material, and as such may be formed in various configurations or shapes, or as will be described, cut into the desired shape. In an embodiment, the device 10 is formed as shown in FIG. 2, wherein a base substrate 12 is formed with an image layer 14 on the top surface thereof. The substrate 12 may be constructed of a suitable material for printing of material thereon, such as paper, plastic or other polymeric materials, metal or metallics, or the like. In an embodiment, the image to form image lager 14 may be acquired by high quality imaging techniques and applied to the substrate 12 by high quality printing procedures. High resolution imaging and printing of the image will provide an appearance which very closely approximates the actual appearance of a finishing material or product. The image may be acquired from an actual product or material using a high resolution imaging system such as a graphics arts scanner or scanning camera, with resolution to provide the desired image quality. The image is then applied to the substrate 12 via high quality printing procedures, such as digital printing techniques, gravure printing, screen printing, offset printing or flexographic printing as examples. The thickness of the substrate 12 may be configured to provide desired structural characteristics in the product sample device 10, and may be formed of one or more layers.

Alternatively, for some finishing materials such as high pressure laminates or veneers for example, the manufacturer may use a printed layer in the product itself which provides color and/or patterns to the final product. The present invention may utilize such printed layer from the manufacturer to serve as or be applied to the substrate 12, rather than independently imaging the finish product or material. The invention then provides for subsequent color and pattern matching to produce a product sample device according to the present invention which very closely simulates the finish product or material. The thickness of the substrate 12 in such an embodiment may also be configured to provide desired structural characteristics in the product sample device 10, and may be formed of one or more layers.

The image layer 14 on substrate 12 may then be overlaminated with a layer 16 of polymeric material, metallic material, or other suitable material. The overlaminate 16 is positioned to overlay at least a portion of the base substrate 12, and typically may be co-extensive with the substrate 12. The overlaminate layer 16 may be formed of any of a variety of suitable materials, such as polyester materials, polypropylene materials, polycarbonate materials, polyolefin materials, ABC or vinyl materials, or any other suitable materials which are generally clear and will not mask the image 14 on the substrate 12. The overlaminate layer 16 may provide various attributes in the product sample device, including the addition of color to properly color match the actual finishing material or product. It has been noted in such materials or products, the ultimate appearance depends on various factors in the final material or product, and problems in properly matching the appearance can be caused by many contributing elements. For example, due to effects of metamerism, where colors may appear similar or differently depending on the lighting conditions under which they are viewed, matching of a product sample device according to the present invention to an actual finishing product or material is provided by matching of the appearance with the actual product using a variety of techniques. In this embodiment, the overlaminate layer 16 may therefore be provided with one or more additional colors to account for any differences between the color appearance of the device 10 as compared to the actual retail finish product or material.

The distinctive appearance of any finish materials may be contributed to by the color and pattern, but also by texture. In embodiments of the invention, such as shown in FIG. 2, the outer surface 18 of the device 10 is treated to provide the desired physical appearance. In this embodiment, the overlaminate layer 16 may be embossed or otherwise surface treated to prepare the outer surface 18 to closely simulate the appearance of the actual finish product or material. The embossing or other surface treatment provides the aesthetic appearance as that of the desired finish product or material, and allows the product to be considered by the consumer based on the actual product characteristics, yet in a form which greatly reduces costs and other problems as noted previously.

Alternatively, it may be possible to provide the desired surface texture and appearance characteristics using a textured or tactile varnish, without the need for the overlaminate layer or coating 16. Such varnishes are known, and may be used to provide the image of the finish product or material, as well as a desired surface texture.

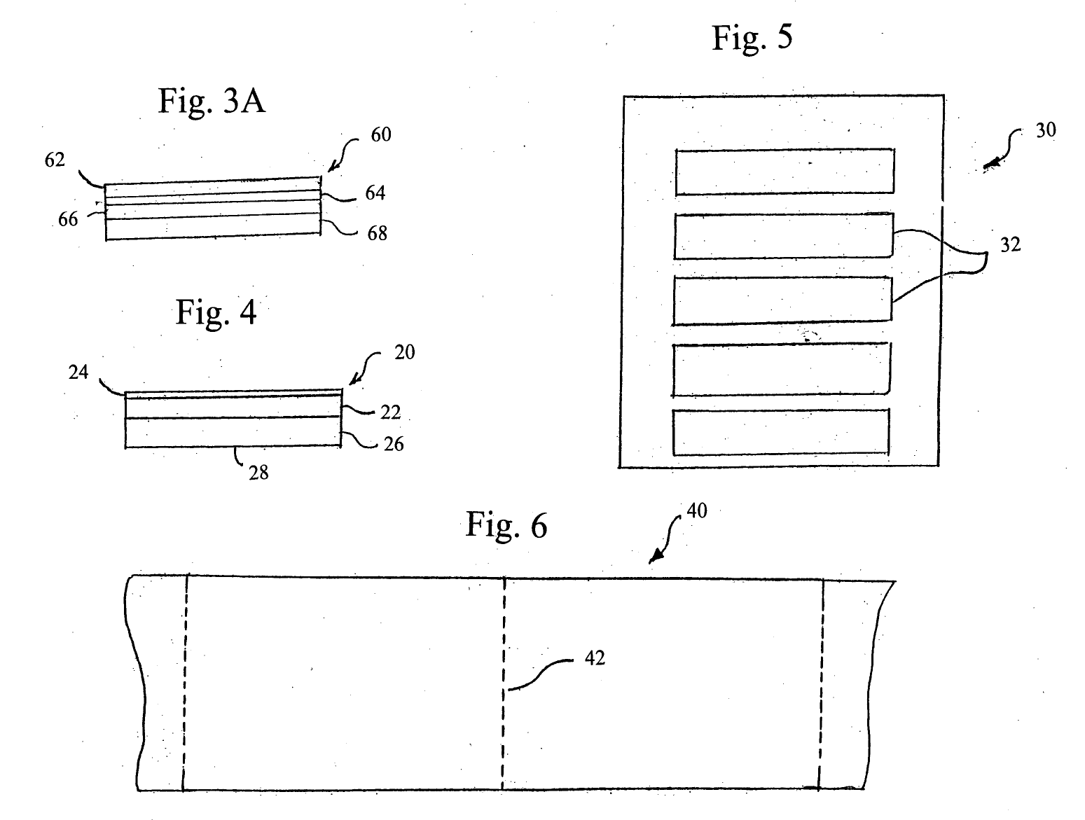

As shown in FIG. 3, in an alternate embodiment of the invention, the device 50 may comprise a layer 52 of printed paper used by the manufacturer to produce the actual finish product or material. A base layer 54 may be provided, to impart a desired additional color to the substantially match the overall appearance of the finish product or material. In an example, the top surface 56 may be printed with a tactile or textured varnish to impart the desired surface appearance which substantially matches the finish product or material. Alternatively, the varnish may itself include one or more colorants to provide the desired overall appearance of the finish product or material, in addition to the desired texture. The base layer 54 may be a printed layer having the proper color characteristics, or a polymeric layer having desired characteristics, or a paper already imparted with a color to provide the desired matching to the finish product or material. Alternatively, if the image layer 52 is provided by imaging of the actual finish product or material, there may be no need to add additional colorants to match the final finish product or material, and the base layer 54 may not be necessary.

In another example, as shown in FIG. 3A, a product sample which has a three-dimensional type of appearance is provided, which could be used to emulate products having such an appearance, such as granite or other stone products or a variety of other products. Further, such an appearance could be used to provide the desired appearance in a sheet type image for a variety of other applications. In this example, the device 60 may comprise an outer or upper layer 62 formed of a clear material, such as a polymer film. The upper surface of the layer 62 may be textured, provide a desired gloss, or have other appearance variables. The clear film layer 62 may be printed on its lower/bottom side with a printed layer 64 having at least one non-opaque area associated therewith. If desired, the film 62 may be directly printed with layer 64 or a separately printed layer 64 may be applied to the back of the film 62. Below the printed layer 64, one or more film layers 66 may be provided. The one or more film layers provide in association with the at least one non-opaque area of the printed layer to form a three-dimensional type of visual effect when viewing from the top of device 60. In an example, the one or more film layers 66 may be a metallized film, providing a metallic appearance through the at least one non-opaque area in the printed layer. This may be used to simulate the appearance of granite products or a variety of other products for example. The film layer 66 may also be provided with a screen of color or other printed materials to provide a desired image appearance. The device 60 may further comprise an optional bottom opaque layer 68, in the event that the one or more film layers 66 do not provide an opaque background. The layer 62, as well as film layer(s) 66 may be of a desired thickness to provide the desired visual appearance of the product. This arrangement increases the depth of viewing certain aspects and areas of the device (the one or more film layers 66 seen through the at least one non-opaque area), through the upper layer 62, providing a three-dimensional type of simulated appearance.

Further, the overlaminate layer or coating 16 of the embodiment of FIG. 2, or other upper surfaces of devices according to the examples herein, may be designed of a material which provides the desired gloss, reflectance or light absorption characteristics to the sample. For product simulation, this appearance would closely approximate the gloss, reflectance or light absorption characteristics of the finish product or material. In the embodiment of FIG. 3, the textured or tactile varnish may be printed on the image layer and provide the desired gloss, reflectance or light absorption properties as desired. The gloss, reflectance or light absorption of a finish product or material is simulated by the overlaminate layer or coating 16, either alone or in conjunction with a bottom layer or coating of material, as will be hereinafter described. The characteristics of the overlaminate layer or coating 16 are chosen accordingly. The invention provides the ability to create an outer layer or coating 16 having the desired surface texture and gloss, reflectance or light absorption characteristics of the finish product or material, and may utilize one or more layers of material if desired. The thicknesses of the layer or layers may also vary depending on the nature of the product and appearance to be achieved.

As another aspect of the present invention, the product sample device 10 according to the invention includes in embodiments the substrate 12 which may be formed of a printable material to allow high quality graphics to be added to the back side 19 of the device 10. A manufacturer could then add significant product information, advertising, directions for use, or any other graphic materials, directly to the product sample device by printing the back side 19 of substrate 12. Presently, to include such information on a product sample produced from the actual finish product or material

In an alternate embodiment, as shown in FIG. 4, the device 20 is provided with a substrate 22 having an image layer or coating 24 provided thereon. The image layer or coating may again be the manufacturer's printed materials for use in preparing the actual product or material, or may be printed from a high quality image of the material as in the embodiment of FIG. 2. In this embodiment, based on the characteristics of the substrate 22 and image 24, visual appearance shifts or other alterations in appearance may occur in the actual finish product or material, based on the backing used in such product or material. In this embodiment, the substrate 22 may be provided with a backing layer 26, which is provided with one or mare colorants to impart the desired final color and appearance to the device 10. The transmittance and reflectance of light through the image layer 24 can alter the appearance of the device 10 and providing the backing layer 26 can be used to compensate for any visual appearance shift from the actual finish product or material. As an example for use in alternate embodiments, the color of a finish product or material may be measured by means of a color spectrophotometer to accurately provide the desired color characteristics. The color of the substrate 22 and/or the substrate 22 and image layer 24 may be similarly ascertained, and the desired color matching is provided by the backing layer 26 by compensating for any mismatch. The backing layer 26 may be formed of a polymeric material, a printed layer with the desired color characteristics or a colored paper layer for example. A polymeric layer may also provide the device 10 with a somewhat tacky back surface 28, which would allow it to be temporarily affixed to a surface for viewing thereof by the consumer. In this regard, the provision of a backing layer, whether including one or more colorants, are contemplated in any of the embodiments of the invention, such as in association with the overlaminate 16 as shown in FIG. 2. Also, in the various embodiments, the provision of embossing or otherwise surface texturing the top layer of device 10 to closely simulate the actual finish product or material is contemplated. This allows for finish materials that otherwise are difficult to display to the consumer, to be effectively provided to the consumer, thereby enhancing sales opportunities. For example, the ability to emboss or add texture the surface of the product sample according to the invention, allows the simulation of soft surfaces such as fabrics like leather or other cloths, some carpets, and the like.

In the sale of finish products or materials, the channels or distribution vary widely, and the present invention is designed to accommodate both the standard approaches to marketing such products or materials, but also new methods of providing access to the architect, designer, builder, consumer or other interested party. As shown in FIG. 5, a display card 30 may be provided, which includes a plurality of individual product samples on a single card 30. The card 30 may be formed similarly to the embodiments of FIGS. 2-4 for example, with a similar color/pattern image layer provided with different surface texturing in individual sample sections 32. With many types of finish products or materials, the manufacturer may offer various surface appearances in association with a standard base product, such as natural, brushed, matte, gloss, sparkle, powdered, etched, quilted or a wide variety of other finishes that change the appearance of the final product. In this embodiment, the invention may provide for easier and more effective comparison of such finishes by the consumer. Alternatively, the individual sample sections 32 may be provided with complimentary image layers with the textures similar or different as desired, or other variations using the same or different product or material images are contemplated to enhance the ability of the consumer to compare and select the desired product or material. It should be evident that the device 10 allows for any type of display type of arrangement as may be desired, affording flexibility in the manufacture of product sample materials while not sacrificing the desired appearance of such samples. Although certain materials, such as paint, have characteristics which may afford similar types of product samples, the finish products and materials to which the invention relates are not conducive to these types of product sample approaches.

In relation to product sample approaches, it would be desirable to allow the user to properly envision the finish product or material in the desired location prior to purchase. The present invention allows for the cost effective manufacture of product samples which meet this desire. As shown in FIG. 6, the product samples according to the invention may also be configured in a manner which allows the user to apply the product samples to the actual surfaces that the actual finish products or materials may be applied. As shown in FIG. 6, the device according to the invention may be configured as a sheet material 40 which is formed in the manner of prior embodiments for example, but dimensioned to allow easy application to standard surfaces, such as countertops, cabinetry, flooring, wall surfaces, or the like. In this embodiment, the sheet material may be formed to have dimensions to match standard countertop surfaces for example, such as having an approximately two (2) foot width, with any length provided, such as a length of three (3) feet. Alternatively, the device 40 may be configured to adapt to typical uses of flooring materials and these spaces, such as in dimensions of three (3) feet by four (4) feet, for example. It should be evident, that based on the finish product or material, the characteristics of the product sample devices according to the present invention can be tailored to meet the user's needs or desires. The invention also allows for large product samples to be provided which allow the user a better example of what the product or material will actually look like in a particular environment as desired.

Additionally, the invention allows for the embodiment of forming sheet materials 40 having perforations 42 at selected lengths, such as at approximately three (3) foot intervals, to provide a standard size with which to cover an existing countertop for example. The ability to perforate the sheet material of device 40 further facilitates shipping of the sheet material in a flat form for distribution of the product samples according to the invention. The number and location of any such perforations can change as desired. The dimensions and configuration of the sheet material 40 may also accommodate use in any other desired location or environment, such as with floor coverings, furnishings or the like. As an example, the device according to the invention may be formed as a simulated leather fabric, and produced in the shape of a cushion associated with the typical chair or sofa, to allow the user to properly envision the finish product or material in the environment they wish. The ability to color match as well as texture match the actual product provides a realistic vies of the product in the consumer's environment.

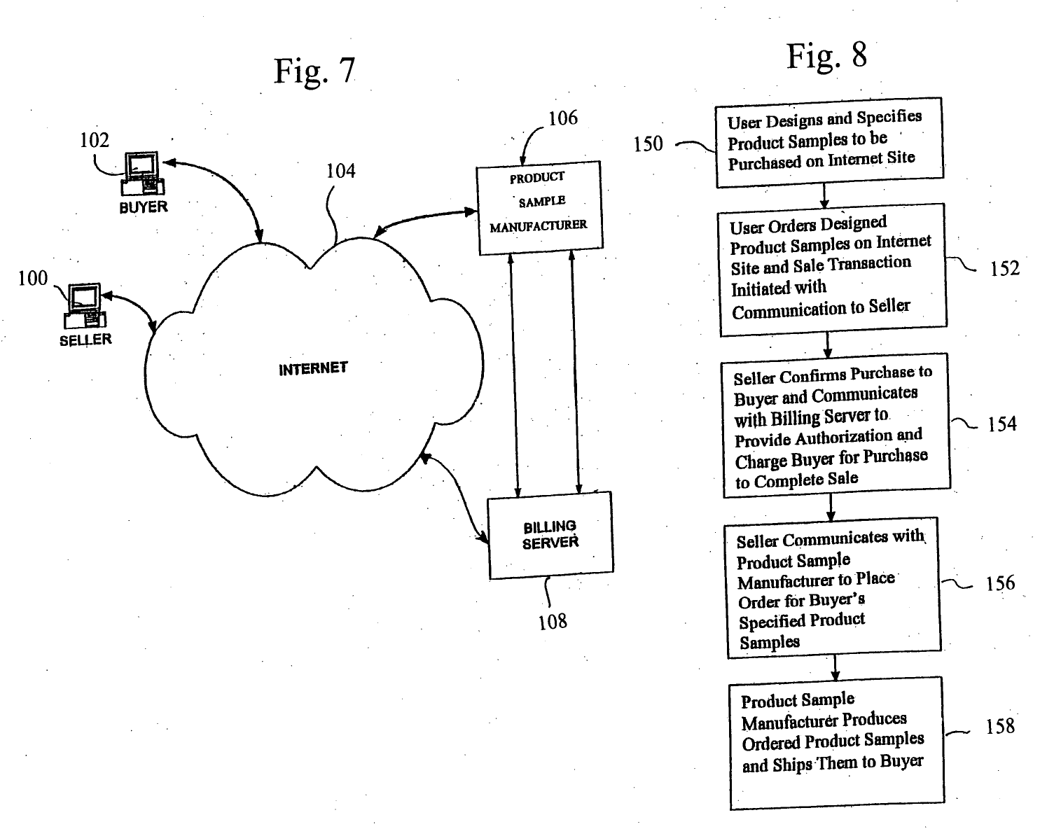

These attributes of the invention also provide for the ability to offer such product samples directly to the consumer for use in this manner. As seen in FIG. 7, a system according to the invention may comprise a seller offering the product sample devices according to the invention via the Internet for example. It should also be recognized that any other desired form of distribution is contemplated, such as mail order, catalogs, retail or the like. The seller may operate a website via a seller computer server 100, and users/consumers can access the website via a buyer computer 102, via the Internet 104. In an example, the seller may also interface with additional resources such a manufacturer 106 of the product sample devices according to the invention. Further, to facilitate sale of the product samples ordered by a consumer, a billing server 108 or service may be used. As shown in FIG. 8, the seller of the product sample devices may provide a website for a potential consumer to review and order product samples as they may desire, correlating to various selected finish products or materials. On the website, the user can select product samples they wish to purchase, or even design product samples to their specific needs, such as to specified widths and lengths, which can then be custom produced per the user specifications, at step 150. Upon selecting product samples for purchase, the user order is submitted to the seller to initiate the sales transaction at 152. The seller may confirm the purchase, and may communicate with the billing server to authorize and obtain payment for the user's purchase at 154, such as via credit card or the like for example. Upon making a sale, the seller can then produce the product samples itself or communicate with a manufacturer to place the buyer's order and initiate manufacture of the purchased product samples at 156. The seller or manufacturer can then prepare the ordered product samples and ship them to the buyer at 158. It should be recognized that the ability to offer high quality product samples in this manner can greatly enhance the user's ability to select desired finish products and materials from respective manufacturers, in a convenient and cost-effective manner. The ability to select desired finish products or materials without requiring product samples formed of the actual products or materials give the individual consumer, as well as the architect, designer or other user, a great amount of flexibility in evaluating the wide variety of finish products and materials available.

While the invention has been illustrated and described in detail in the foregoing drawings and description, the same is to be considered as illustrative and not restrictive in character, it being understood that only illustrative embodiments thereof have been shown and described and that all changes and modifications that come within the spirit of the invention are desired to be protected. Additional features of the invention will become apparent to those skilled in the art upon consideration of the description. Modifications may be made without departing from the spirit and scope of the invention.

What is claimed is:

1. A device for assisting in the selection of finish products or materials comprising,
a base substrate having an image of a finish product or material provided thereon;
a layer of material provided in association with the base substrate which provides a predetermined visual appearance shift in the image associated with the base substrate to make a laminate, and
a surface texture formed on the top surface of the laminate to form a product sample which closely simulates a predetermined actual finish product or material.

2. A method for providing product samples to a user, comprising the steps of:
providing information regarding product sample devices on an Internet site,
providing via the site the ability for the user to review and select one or more product samples according to desired specifications and characteristics on the site,
providing the user the ability to order and purchase specified product samples on the site so as to be a buyer,
transmitting billing information associated with the buyer for authorizing and transferring funds needed for the purchase of the ordered product samples, and
manufacturing and/or filling the buyers order and shipping the ordered product samples to the buyer.
